(19) United States
(12) Patent Application Publication
(10) Pub. No.: US 2025/0032758 A1
(43) Pub. Date: Jan. 30, 2025

(54) NEEDLE ASSEMBLIES AND SAFETY CATHETER ASSEMBLIES WITH BLOOD DIVERTING FEATURE

(71) Applicant: SMITHS MEDICAL ASD, INC., PLYMOUTH, MN (US)

(72) Inventors: Thomas T Koehler, Pelzer, SC (US); Christopher D Roehl, New Hartford, CT (US); Jonathan Sanborn, St Louis Park, MN (US); Dean SIbik, Harris, MN (US)

(73) Assignee: SMITHS MEDICAL ASD, INC., PLYMOUTH, MN (US)

(21) Appl. No.: 18/715,143

(22) PCT Filed: Dec. 1, 2022
(86) PCT No.: PCT/US2022/051467

§ 371 Date: May 31, 2024

(30) Foreign Application Priority Data
Dec. 8, 2021 (US) 63287331

(51) Int. Cl. A61M 25/06; A61M 25/00

(52) U.S. Cl. CPC A61M 25/0693; A61M 25/0097; A61M 25/0606; A61M 25/0637; A61M 2205/583; A61M 2206/10

(57) ABSTRACT

A fluid or blood diverter is provided in the needle housing of a needle assembly for redirecting the inflowing blood to a distal section of the fluid diverter that may be enclosed by the distal portion of the needle housing so that a clinician can readily view the blood flash without having to look back to the flashback chamber at the proximal portion of the needle housing. At the distal section, the inflowing blood is redirected to the flashback chamber. The needle housing with the blood diverter is adapted to be used in safety catheter needle assemblies that may include a passive needle safety control mechanism including a clip guard. Different ways of coupling the needle housing and the clip guard with catheter hub assemblies with and without passive blood control are also described.

### FIELD OF THE INVENTION

The present invention relates to needle assembly devices including safety catheter assemblies that have passive blood control and mechanism for shielding the sharp distal tip of the needle from the time that the devices are ready to use to the disposal of the devices, and a blood divert mechanism adapted to be incorporated into the devices that enables the user to determine that the needle is correctly placed into the vasculature of the patient.

### BACKGROUND OF THE INVENTION

Needle assembly devices conventionally include a needle having a distal end including a sharp distal tip and a proximal end that is connected to a needle hub. The lumen or through passage of the needle is in alignment with a passageway through the needle hub so that when the distal end of the needle is correctly inserted into the vein or artery of the patient, blood under pressure would pass through the needle lumen and the passageway of the needle hub into a blood store, for example a syringe. The proximal end of the needle may also be flush with the proximal end of the needle hub such that the passageway of the needle hub is the proximal portion of the lumen. In the case of a catheter assembly such as a peripheral intravenous catheter (PIVC), the needle hub may be a part of a needle housing that has a flashback chamber at its proximal portion whereinto the blood flows. Blood in the flashback chamber provides an indication to the user or clinician that the distal end of the needle, and the distal end of the catheter that overlies the needle, are correctly placed into the vasculature of the patient.

Some of the catheter assemblies have passive mechanisms that prevent the sharp distal tip of the needle from being exposed, when the needle is withdrawn from the patient. Such safety catheter assemblies are described in co-pending applications 63/227,497 entitled “Safety Catheter Assembly With Blood Control”, 63/227,508 entitled “Safety Catheter Assembly’, and Ser. No. 63/227,520 entitled “Catheter Assembly With Needle Tip Protection and Passive Blood Control”, all filed on Jul. 30, 2021. The respective disclosures of the '107, '520 and '508 applications are incorporated by reference to the disclosure of the present application.

For the needle assembly and the safety catheter assembly with a needle housing, the needle hub is formed at a distal portion and the flashback camber is at the proximal portion of the housing. When a clinician inserts the needle into the patient, she focuses to the tip of the needle being inserted into the patient. To ensure that the needle is placed correctly into the vein or artery of the patient, the clinician would have to take eyes off the tip of the needle and look at the back of the needle housing to see whether blood is flowing into the flashback chamber. Oftentimes, the flashback chamber is obstructed from viewing by the hand of the clinician.

Therefore, for the clinician to readily determine that the distal end of the needle is correctly placed into the vasculature of the patient without having to take her eyes off the needle of the device, means needs to be provided to the device to enable the clinician to see the blood flow without having to look to the flashback chamber at the back of the needle housing, and/or move her hand to a position where the flashback chamber is not obstructed from view.

### SUMMARY OF THE PRESENT INVENTION

To enable the clinician to readily view the flow of blood in the needle housing, at the outlet of the passageway of the needle hub that connects to the lumen of the needle a fluid diverter is positioned for receiving the blood output from the passageway of the needle hub. The diverter is configured to have a fluid path that guides the blood to flow in the distal direction to a distal portion of the needle housing where the clinician can readily see the blood flow. The distal portion of the needle housing where the blood flow is guided may have a transparent or translucent viewing window pane such that the blood can be readily seen. At the distal portion, the fluid path is divided into at least two fluid paths that guide the blood to flow in the proximal direction into the proximal portion of the needle housing that is the flashback chamber. As the portion of the needle housing that makes up the flashback chamber is transparent or translucent, when blood reaches the flashback chamber, the clinician has further confirmation that the distal end of the needle has been correctly placed into the patient. A flash pug is fitted to the open proximal end of the needle housing to close off the flashback chamber to enable air to pass through but prevents blood or other fluids from passing out of flashback chamber.

The fluid diverter is configured such that it has distal surface that abuts against the proximal end surface of the needle hub, with the passageway from the needle hub in alignment with the bore that forms the fluid path inside the diverter. The needle housing may be configured to have a space between the needle and the wall of the needle housing such that a distal extension portion of the diverter may be fitted into the space. The distal extension portion contains the distal part of the distal fluid path that redirects the blood from the needle hub to a collection well or section at the distal portion of the needle housing. The distal extension of the diverter, and the window of the needle housing that overlies the distal extension of the diverter, may be formed from transparent medical plastics to enable the clinician to readily see the blood flow to the distal extension. At the collection well, the distal fluid path is divided into two proximal directional fluid paths that lead into the flashback chamber at the proximal portion of the needle housing.

The fluid diverter may be fitted to conventional needle assemblies in which the contaminated needle, once removed from the patient, is recapped by a sheath. The diverter is moreover adapted to be a part of a safety catheter assembly where the needle housing is provided with a connector mechanism that enables it to connect to a catheter hub that may have a blood control mechanism such as a septum of a multi-use seal member, along with a passive safety mechanism that prevents the contaminated sharp needle tip from being exposed to the environment when the needle is removed from the patient. In the case of the safety catheter assembly, the needle housing may have a needle guard or collar that houses a needle tip protector described in the above incorporated by reference '497, '508 and '530 applications. The collar or needle guard is slidable along the shaft of the needle and may be separable from the needle housing to prevent the distal sharp tip of the needle from being exposed when the needle is withdrawn from the patient.

The present invention is therefore directed to an apparatus comprising a needle housing having a needle hub with a passageway, a needle having a sharp distal end and a proximal end connected to the needle hub, the needle having a lumen extending from the sharp distal end to the proximal end in alignment with the passageway of the needle hub, a rear chamber in the needle housing proximal the needle hub adapted to receive fluid that enters the distal end of the needle, and a fluid diverter in fluid communication with the needle hub for receiving the fluid incoming from the lumen and redirecting the fluid to flow to a distal section of the fluid diverter viewable through a distal portion of the housing.

The present invention is also directed to a method of establishing blood flow in a needle housing, comprising the steps of: providing a needle housing including a distal portion having a transparent or translucent viewing part; attaching a proximal end of a needle having a lumen and a sharp distal end to a needle hub integrally formed inside a distal portion of the needle housing such that the lumen of the needle is in alignment with a passageway of the needle hub to establish a fluid passage for receiving blood that enters the distal end of the needle when the needle is placed into a vein or artery of a patient; providing a flashback chamber in the needle housing proximal the needle hub; and positioning a fluid diverter adjacent the needle hub for receiving the blood output from the passageway, the fluid diverter redirecting the blood to flow in a distal direction to the transparent or translucent viewing part of the distal portion of the housing where a user can observe the blood flow.

The present invention is further directed to an apparatus, comprising: a needle housing including a proximal portion, a distal portion having a transparent or translucent viewing part and a flashback chamber at the proximal portion; a needle having a lumen, a sharp distal end and a proximal end attached to a needle hub integrally formed inside a distal portion of the needle housing such that the lumen of the needle is in alignment with a passageway of the needle hub to establish a fluid passage for receiving blood that enters the distal end of the needle when the needle is placed into a vein or artery of a patient; and a fluid diverter positioned adjacent the needle hub for receiving the blood output from the fluid passage, the fluid diverter redirecting the blood to flow in a distal direction to a distal section of the fluid diverter located in the transparent or translucent viewing part of the distal portion of the housing where a user can observe the blood flow.

The present invention is furthermore directed to a catheter assembly comprising: a catheter hub having a distal portion and a proximal portion having an open proximal end, the proximal portion having an internal cavity defined by a wall between the distal portion and the open proximal end; a catheter extending from the distal portion of the catheter hub; a seal member positioned in and axially shiftable along the internal cavity of the catheter hub, the seal member being a substantially cylindrical wall having an inner surface defining a bore, the seal member including a membrane for partitioning the bore to have at least a hollow proximal section; a needle housing having a distal portion, a proximal portion, an integral needle hub at the distal portion and an open distal end; a needle having a proximal end attached to the needle hub and a sharp distal tip, the needle extending through the catheter and slidably extending along the catheter with the distal tip extending beyond a distal end of the catheter in a ready position; a needle tip protector slidably positioned along the needle having at least one barrier, at least one portion of the needle tip protector being biased by the needle to engage the seal member when the catheter assembly is in the ready position; a fluid diverter in abutment with the needle hub in the needle housing for receiving fluid incoming from the needle and redirecting the fluid to flow to a distal section of the fluid diverter viewable through the distal portion of the needle housing.

The present invention further relates to a catheter assembly comprising: a catheter hub having a body including a wall, a distal end, an open proximal end and an interior cavity defined between the distal end, the proximal end and an inner surface of the wall; a catheter having a distal end extending from the distal end of the catheter hub to establish a passage to the interior cavity; a port integrally extending in a substantially upright direction from the catheter hub, the port having a through bore to the interior cavity of the catheter hub; a sleeve member in circumferential contact with the inner surface of the wall of the catheter hub for sealing the bore; a needle housing including a distal portion having an open distal end and a needle hub inside the distal portion; a needle having a proximal end attached to the needle hub and a sharp distal tip, the needle extending pass the interior cavity of the catheter hub and slidably extending along the catheter with its sharp distal tip extending beyond the distal end of the catheter when the catheter assembly is in a ready state; and a fluid diverter in abutment with the needle hub in the needle housing for receiving fluid incoming from the needle and redirecting the fluid to flow to a distal section of the fluid diverter viewable through the distal portion of the needle housing.

The present invention yet further relates to a catheter assembly comprising: a catheter hub having a body including a wall, a distal end, an open proximal end and an interior cavity defined between the distal end, the proximal end and an inner surface of the wall; a catheter having a distal end extending from the distal end of the catheter hub to establish a passage to the interior cavity; an upright port integrally extending from the catheter hub, the port having a through bore to the interior cavity of the catheter hub; a sleeve member in circumferential contact with the inner surface of the wall of the catheter hub for sealing the bore; a needle housing including a distal portion having an open distal end and a needle hub inside the distal portion; a needle having a proximal end attached to the needle hub and a sharp distal tip, the needle extending through the interior cavity of the catheter hub and slidably extending along the catheter with its sharp distal tip extending beyond the distal end of the catheter when the catheter assembly is in a ready state; a needle tip protector slidably positioned along the needle, the needle tip protector having a base and opposing arms extending distally from the base having corresponding barriers and apexes; and wherein when the catheter assembly is in the ready state, the needle passes between the barriers so that the apexes of the arms are biased into frictional contact with a locking groove or protuberance at the inner surface of the wall of the catheter hub.

The present invention is yet furthermore directed to a catheter assembly comprising: a catheter hub having a body including a wall, a distal end, an open proximal end and an interior cavity defined between the distal end, the proximal end and an inner surface of the wall; a catheter having a distal end extending from the distal end of the catheter hub to establish a passage to the interior cavity; a sideport integrally extending from the catheter hub having a through bore to the interior cavity of the catheter hub; a septum positioned in the interior cavity proximal the through bore of the sideport; a needle housing including a distal portion having an open distal end and a needle hub inside the distal portion; a needle having a proximal end attached to the needle hub and a sharp distal tip, the needle extending pass the septum into interior cavity of the catheter hub and slidably extending along the catheter with its sharp distal tip extending beyond the distal end of the catheter when the catheter assembly is in a ready state; and a fluid diverter positioned in the needle housing for receiving fluid incoming from the needle from the needle hub, the fluid diverter redirecting the fluid to flow to a distal section of the fluid diverter viewable through the distal portion of the needle housing.

The present invention moreover relates to a catheter assembly, comprising: a catheter hub having a body including a wall, a distal end, an open proximal end and an interior cavity defined between the distal end, the proximal end and an inner surface of the wall; a catheter having a distal end extending from the distal end of the catheter hub to establish a passage to the interior cavity; a sideport integrally extending from the catheter hub having a through bore to the interior cavity of the catheter hub; a septum positioned in the interior cavity proximal the through bore of the sideport; a needle housing including a distal portion having an open distal end and a needle hub inside the distal portion; a needle having a proximal end attached to the needle hub and a sharp distal tip, the needle extending pass the septum into interior cavity of the catheter hub and slidably extending along the catheter with its sharp distal tip extending beyond the distal end of the catheter when the catheter assembly is in a ready state; a septum retainer having a wall, an internal cavity defined by the inner surface of the wall, a distal surface and a proximal surface fixedly positioned in the interior of the catheter hub, the distal surface in abutment with a proximal surface of the septum to prevent proximal movement of the septum; and a needle tip protector slidably positioned along the needle, the needle tip protector having a base and opposing arms extending distally from the base to respective opposing barriers and apexes, at least the barriers and apexes of the arms of the needle tip protector are positioned in the internal cavity of the septum retainer in a ready position wherein the barriers are biased by the needle to move the apexes into engagement with the inner surface of the wall of the retainer to non-removably hold the needle tip protector to the septum retainer.

### BRIEF DESCRIPTION OF THE FIGURES

The present invention will become apparent and the invention itself will be best understood with reference to the following description of the invention taken in conjunction with the accompanying drawings, wherein:

FIG. 1 shows an exemplar safety catheter assembly in a ready to use position, with a needle protection sheath covering the needle before use, and the sheath removed for use;

FIG. 2 is a cross-sectional view of the safety catheter assembly of the instant invention with an inventive blood diverter;

FIG. 3 is an enlarged view of the catheter hub of the FIG. 2 safety catheter assembly shown with its proximal luer end coupled to a connector of a needle guard of the needle housing;

FIG. 4A is a side view of the safety catheter assembly of FIG. 2;

FIG. 4B is a cross-sectional side view of the FIG. 4A safety catheter assembly;

FIG. 4C is a cross-sectional top view of the FIG. 4A safety catheter assembly;

FIG. 5 is a semi-transparent view of the distal portion of the needle housing with an exemplar blood diverter of the instant invention operationally in fluid communication with the integral needle hub of the needle housing;

FIG. 6 is a semi-transparent perspective view showing the needle hub and the blood diverter, and the fluid paths of the blood diverter in fluid communication with the needle hub and the flashback chamber of the needle housing;

FIG. 7 shows the assembly of the blood diverter into the needle housing through the flashback chamber;

FIG. 8A shows the needle tip protector guard or clip guard of the needle housing coupled to the catheter hub with the needle housing moved proximally away from the catheter hub;

FIG. 8B shows the clip guard separated from the catheter hub;

FIG. 9A is a cross-sectional side view of the safety catheter assembly in a safe position with the blood diverter shown in the needle housing;

FIG. 9B is a cross-sectional plan view of the safety catheter assembly showing the proximal outlet of the needle hub connected to and in fluid communication with the blood diverter;

FIG. 10A is a cross-sectional top view showing a safety catheter assembly with a multi-use seal member and the collar of the clip guard being fixedly coupled to the catheter hub by way of the interaction between the needle tip protector clip and the seal member;

FIG. 10B shows the needle in a safe position with the clip guard and the catheter hub in the process of separating from each other;

FIG. 10C shows the needle in a safe position inside the needle protection clip in the clip guard, and the clip guard having separated from the catheter hub;

FIG. 11A shows the catheter hub and a luer connector;

FIG. 11B is a cross-sectional view of the luer connector and the catheter hub, showing the seal member and actuator inside the catheter hub;

FIG. 11C is a cross-sectional view showing the luer connector coupled to the catheter hub, and the biasing of the membrane of the seal member against the proximal open end of the actuator to establish a fluid path through the catheter hub and the luer connector;

FIG. 12A is an illustration of a safety catheter assembly that has an upright sideport catheter hub;

FIG. 12B shows the FIG. 12A safety catheter assembly with the sheath having the luer lock cap removed from the needle;

FIG. 13A is a cross-sectional view of the safety catheter assembly of FIG. 12B with a blood diverter;

FIG. 13B is an enlarged view of the upright sideport catheter hub of the FIG. 13A safety catheter assembly;

FIG. 14A is a side view of the safety catheter assembly with the upright sideport catheter hub in the ready position;

FIG. 14B is a cross-sectional view of the FIG. 14A safety catheter assembly showing the blood diverter in the needle housing;

FIG. 14C is a cross-sectional plan view of the FIG. 14A safety catheter assembly with the blood diverter;

FIGS. 15A and 15B are illustrations of a closed system catheter (CSC) safety catheter assembly;

FIG. 16A shows the CSC safety catheter assembly in a ready to use position;

FIG. 16B shows the CSC safety catheter assembly in a position where the winged needle is in an advancing position;

FIG. 16C shows the CSC safety catheter assembly in the fully advanced position;

FIG. 16D shows the CSC safety catheter assembly with the needle assembly separated from the catheter hub assembly;

FIG. 17A is a cross-sectional top view of the CSC safety catheter assembly showing a distal section of the blood diverter in fluid communication with the needle hub passageway in the needle housing;

FIG. 17B is an enlarged cross-sectional top view of the catheter hub and the clip guard of the CSC safety catheter assembly of FIG. 17A;

FIG. 18A is a perspective view of the catheter hub assembly of the CSC safety catheter assembly;

FIG. 18B is a disassembled view of the different components of the catheter hub assembly of the CSC safety catheter assembly; and

FIG. 18C is a cross-sectional view of the catheter hub of the CSC safety catheter assembly.

### DETAILED DESCRIPTION OF THE INVENTION

For the discussion below, it should be appreciated that the term “distal,” as used herein, refers to the direction along an axis that lies parallel to a needle cannula of a safety catheter assembly that is closest to the subject during catheter insertion. Conversely, the term “proximal,” as used herein, refers to the direction lying along the axis parallel to the needle cannula that is further away from the subject when the catheter is inserted into the vein of the subject, opposite to the distal direction. Also, a needle cannula may simply be referred to as a needle and the through bore of the needle may be referred to as the cannula or lumen. Other terms that may also be used to refer to the distal end and proximal end of the safety catheter device may include “patient end” and “user end”, respectively. Also, for the sake of simplicity, a safety catheter assembly device, or PIVC, may simply be referred to as “catheter assembly” or “safety catheter”, the needle tip protector as “safety needle clip” or simply “safety clip”, and the guard housing that has a collar that overlies the proximal portion of the catheter hub and isolates the safety clip from the environment after the safety clip is removed from the catheter hub as “clip guard”. it should further be noted that the fluid diverting feature of the invention may also be used with needle assemblies that are not safety catheter devices that have clip guards.

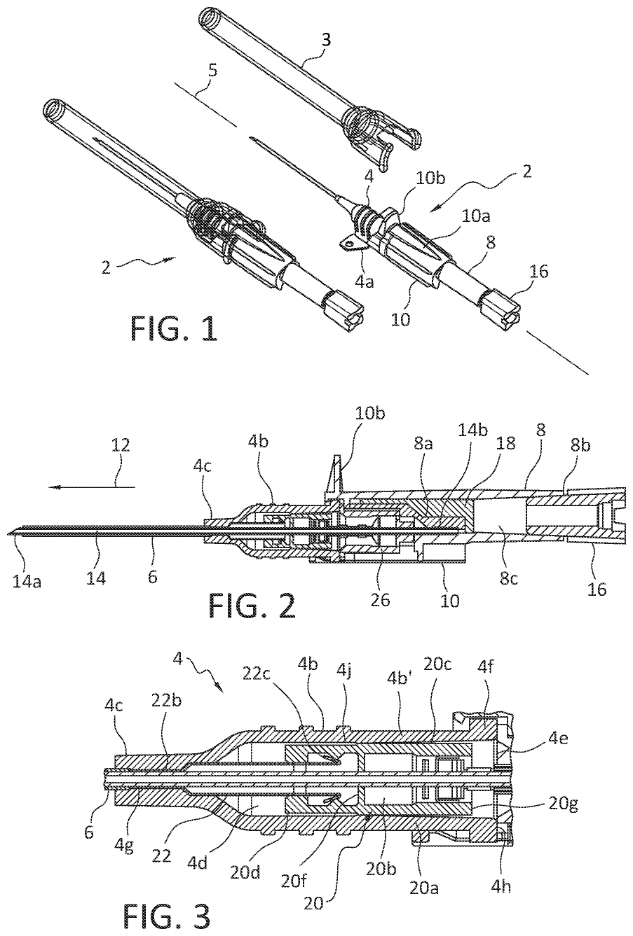

FIG. 1 shows a safety catheter assembly 2 that has a blood control mechanism with a straight wing catheter hub. As shown, safety catheter assembly 2 has a catheter hub 4 that for the FIG. 1 embodiment has wings 4a. Catheter hub 4, as shown in the cut-away view of FIG. 2 and in the enlarged cross-sectional view of FIG. 3, has a main body portion 4b and a distal portion 4c to which a catheter 6 is press fitted as is known conventionally. Main portion 4b has a wall 4b′ that defines an internal or interior cavity 4d. Catheter hub 4 further has an open proximal end 4e whereat a circumferential or a non-continuous semi-circumferential flange or rib 4f is provided for connection to the needle housing, more particularly to the collar of the needle protection guard or clip guard of the needle housing as will be discussed in more detail, infra.

The safety catheter assembly 2 moreover has a needle housing 8 that has a gripping portion or member 10, which may simply be referred to as the gripper of needle housing 8. As shown in FIG. 1, gripper 10 is a rounded or semi-circular shell that, except for the substantially flat window 10a at the top of the distal portion of the needle housing, integrally extend from and at least surround the sides of the distal portion of the needle housing 8 to enable a user to readily grasp the safety needle assembly 2. The substantially planar window 10a may be a transparent or translucent pane that enables the user to readily look into the distal portion of needle housing 8. A push tab 10b is provided at the distal end of gripper 10 to provide a place for the user or clinician to push or advance the safety catheter assembly 2 in the distal direction, as indicated by distal directional arrow 12, to insert the sharp distal tip 14a of the needle 14 and the catheter 6 that overlies needle 14 into the patient. Needle 14 has a proximal end 14b attached to a needle hub 8a integrally formed in the distal portion of needle housing 8.

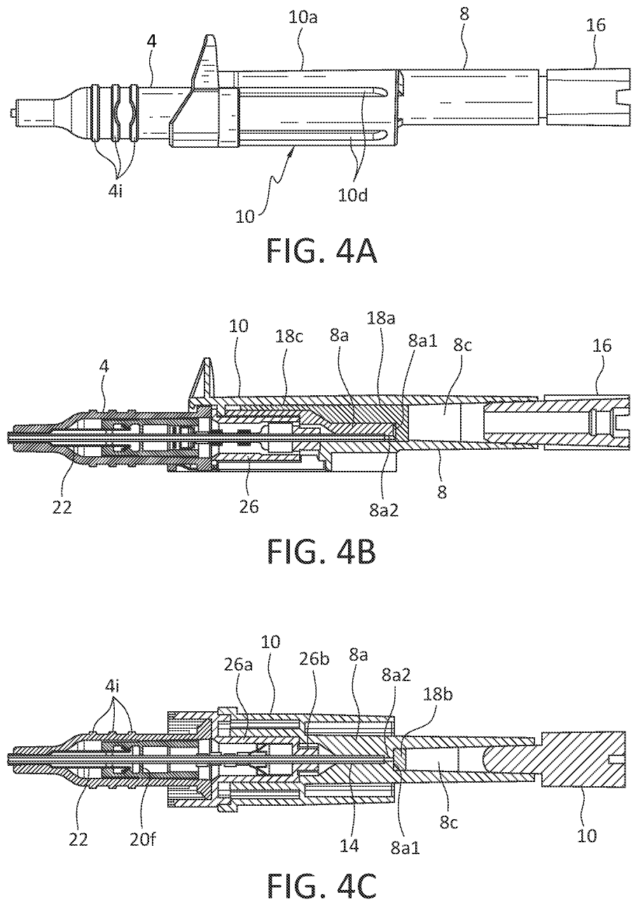

With reference to FIGS. 2 and 4A-4C, needle housing 8 is shown to have an open proximal end 8b fitted with a flash plug 16 such that an interior cavity or flashback chamber 8c is formed at the proximal portion of needle housing 8. As is conventionally known, flash plug 16 may include a hydrophilic material that allows air to pass but prevents fluid such as blood from passing. Also as conventionally known, needle housing 8 may be made from transparent or translucent medical grade plastics such that blood in the interior of flashback chamber 8c may readily be viewed by the clinician as a means to confirm that the distal end of the needle is correctly placed into the vasculature, i.e., the blood vessels including the vein or artery, of the patient.

For the safety catheter assembly 2 of the instant invention, a fluid diverting mechanism for example a fluid or blood diverter 18, is provided at the proximal end 8a1 of needle hub 8a. As is conventionally known, needle 14 is hollow and the passage therethrough may be referred to as the lumen of the needle. Needle hub 8a has a passageway 8a2 in alignment with the lumen of needle 14 which proximal end is attached to needle hub 8a by any number of conventional means such as press fitting or gluing. It should be appreciated that the proximal end of the catheter may be flush with the proximal end of the passageway 8a2 such that the lumen of the needle forms the fluid passage that extends from the distal tip of the needle to the proximal end of needle hub 8a. In FIG. 4C, the proximal end of needle 14 is shown not to be flush with the open proximal end of needle hub 8a so that passageway 8a2 is in alignment with the lumen of needle 14 to establish the fluid passage from the distal end of needle 14 to the proximal open end of needle hub 8a.

As shown in FIGS. 2 and 4B-4C, fluid or blood diverter 18, blood diverter henceforth, is in fluid communication with passageway 8a2 of needle hub 8, such that the fluid or blood output from passageway 8a2 is intercepted and received by blood diverter 18. In particular, the incoming blood from the needle is received by an input bore of blood diverter 18. Blood diverter 18 functions to redirect the incoming blood distally to a distal section 18e of the blood diverter enclosed by distal front portion of needle housing 8 and viewable through the needle housing via its transparent or translucent window pane. The incoming blood is next redirects proximally to the flashback chamber 8c.

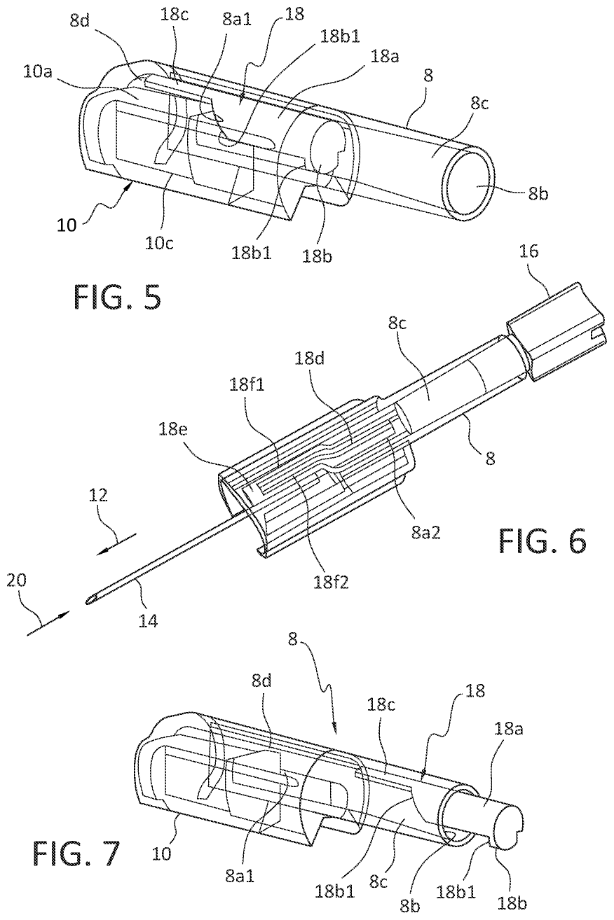

In FIGS. 5-7, blood diverter 18 is illustrated as a one piece component that has a proximal end section 18b that may be positioned adjacent proximal end 8a1 of needle hub 8a such that the distal surface of proximal end section 18b of blood diverter 18 is in abutment with the surface of proximal end 8a1, with the inlet of blood diverter 18 aligned in line with passageway 8a2 of needle hub 8a so that blood from the needle flows into the inlet bore which becomes the distal direction fluid path 18d of blood diverter 18. As best shown in FIG. 6, the inlet bore of blood diverter 18 may be considered as being the portion of fluid path 18d that meets passageway 8a2 and thus is in fluid communication with the lumen of the needle to receive the incoming fluid from the needle, when the needle is correctly placed into the vein or artery of the patient.

Further with reference to FIGS. 5-7, blood diverter 18 has a main portion 18a, a proximal end section 18b and a distal extension section 18c. As best shown in FIG. 6, fluid such as blood flows into needle 14 as per shown by directional arrow 20, through passageway 8a2 of needle hub 8 into blood diverter 18 at its proximal end section 18b, the blood is redirected by distal fluid path 18d in the distal direction as per shown by directional arrow 12 to a distal blood collection well or section, or simply distal section 18e, of the blood diverter. At distal section 18e the fluid path is divided into two proximal direction fluid paths 18f1 and 18f2 that fed into flashback chamber 8c.

FIG. 5 shows the distal surface 18b1 of proximal end section 18b of blood diverter 18 being in abutment with the proximal end surface 8a1 of needle hub 8a in needle housing 8. FIGS. 4B-4C show the blood flows from the outlet of passageway 8a2 of needle hub 8 into the inlet of proximal end section 18b of blood diverter 18. As shown in FIG. 6, the blood is redirected by distal fluid path 18d in the distal direction to distal section 18e. From distal section 18e, the two proximal direction fluid paths 18f1 and 18f2 redirect the blood in the proximal direction to flashback chamber 8c.

FIG. 7 shows blood diverter 18 being inserted into needle housing 8 by way of proximal end 8b and through interior cavity or flashback chamber 8c of needle housing 8. There is a distal space 8d in the distal portion of needle housing 8 defined by top pane 10a of gripper 10 and needle hub 8a for receiving distal extension section 18c of blood diverter 18, when blood diverter 18 is fully inserted into needle housing 8 such that its distal surface 18b1 of proximal end section 18b is in abutment contact with the surface of proximal end 8a1 of needle hub 8a to establish a fluid communication path between passageway 8a2 of needle hub 8 and the inlet of blood diverter 18, and from there to distal direction fluid path 18d. It should be noted that, as discussed above, if the open proximal end of needle 14 is flush with the open proximal end of needle hub 8, then it is the lumen of the needle that establishes the fluid communication path with the inlet of blood diverter 18.

With the top window or pane 10a of gripper 10 being transparent or translucent, the clinician does not need to divert her vision to the flashback chamber 8c to see the presence of blood. Instead, the clinician can readily view the presence of blood in distal section 18e, as well as the blood flow along proximal fluid paths 18f1 and 18f2. Thus, without having to remove her hand and move her head, the clinician can, by means of the presence of blood displayed in distal section 18e, and also in fluid paths 18f1 and 18f2, confirm that the distal end of the needle 14 is correctly placed into the vasculature of the patient. Longitudinal ribs 10d provided along gripper 10 assist the clinician with the griping and maneuvering of the safety needle assembly 2. Although a safety catheter assembly is shown to illustrate the blood diverter in the needle housing, it should be appreciated that the blood diverter can also be provided to a needle housing of a needle assembly that does not operate with a catheter hub assembly, or that the needle has slidably coupled thereto a passive safety mechanism.

With reference to FIGS. 2, 3 and 4B-4C, catheter hub 4 of the safety catheter assembly 2 is shown to include a main body portion 4b which wall 4b′ defines an interior cavity 4d, a distal portion 4c having an aperture 4g whereat the proximal end of the catheter 6 is fixedly coupled, and an opened proximal end 4e whereat one or more threading ribs or flange 4f are provided to enable the catheter hub to be threadingly mated to counterpart connecters of syringes, injectors, pumps or the like as is conventionally known. The combined end surfaces of proximal end 4e and the flange 4f form a proximal contact surface 4h. A plurality of splines 4i circumferentially formed along the outer wall surface of main body portion 4b may be provided to enable a user to more readily grasp the catheter hub. A groove or step 4j may be provided circumferentially about the inner surface of wall 4b′ to secure a seal member in the interior cavity 4d of catheter hub, as described below.

Further with reference to FIGS. 3 and 4B-4C, a valve or seal member 20 is provided or positioned in and axially shiftable along the interior cavity 4d of catheter hub 4. Seal member 20 may be made from an elastomeric material that is generally flexible and may include suitable materials such as, for example, silicone or polyisoprene. In one embodiment, seal member 20 may be formed as a unitary or monolithic member through various molding processes including, for example, injection molding processes generally known in the art.

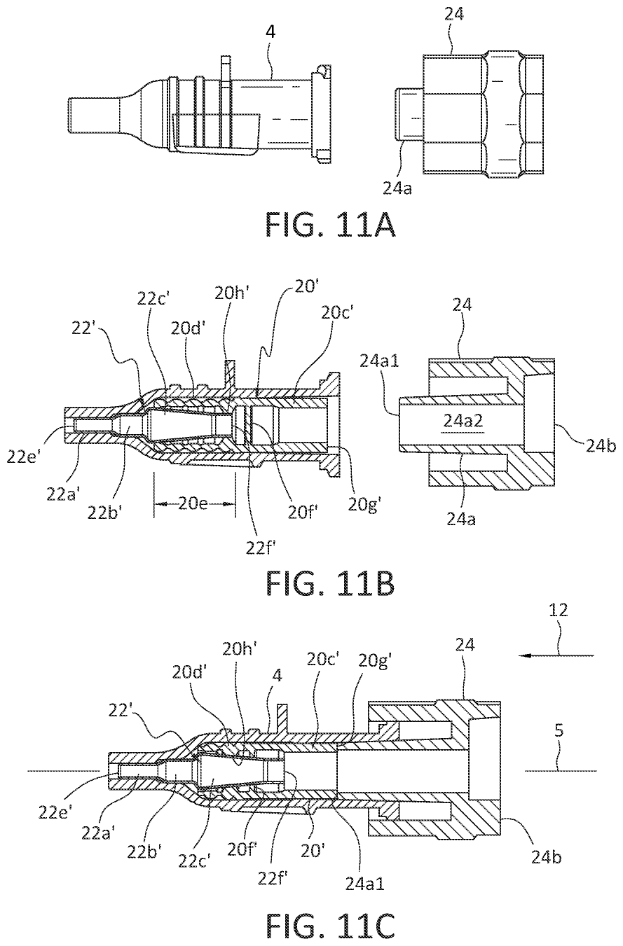

As shown, seal member 20 has a substantially cylindrical shaped wall 20a that defines a bore 20b. Seal member 20 includes a proximal cylindrical portion 20c and a distal portion 20d. The portion of wall 20a that defines proximal portion 20c is substantially uniform along its length. Although not shown in the embodiment of FIGS. 3 and 4B-4C, the portion of wall 20a that defines distal portion 20d may have a corrugated shape that may be in the shape of an accordion along length of its distal portion, designated 20e in the embodiment of FIG. 11B. An integral membrane 20f partitions bore 20b between proximal portion 20c and distal portion 20d. Although shiftable axially, seal member 20 is non-removably secured to the interior cavity 4d of catheter hub 4, as a circumferential flange 20g at approximately the juncture between proximal portion 20c and distal portion 20d catchingly engages the step 4j at the interior cavity 4d of catheter hub 4. Step 4j prevents seal member 20 from proximal movement out of catheter hub 4 once seal member 20 is fittingly inserted and seated into the interior cavity 4d of catheter hub 4 by way of opened proximal end 4e of catheter hub 4.

For the embodiment shown in FIGS. 3 and 4B-4C, inside interior cavity 4d distal of membrane 20f is an actuator 22 that has its distal end 22b frictionally held to the aperture 4g at the distal portion 4c of catheter hub 4. Actuator 22 shown in FIG. 3 has a barb 22c at its proximal end for holding membrane 20f of seal member 20 open when the seal member 20 is impacted by a distal force as per described in U.S. Pat. No. 8,652,104, assigned to the same assignee of the instant invention. The disclosure of the '104 patent is incorporated by reference in its entirety to the instant application.

For the embodiment shown in FIGS. 11A-11C, the elastomeric seal member 20′ has an accordion shaped distal portion 20d′ that is compressible when proximal portion 20c′ is under pressure or is impacted by a distal driving force at proximal end 20g′. As distal portion 20d′ is elastomeric, distal portion 20d′ would return to its natural position when the pressure or impact force is removed from proximal end 20g′. FIGS. 11B-11C further show an actuator 22′ that is configured differently from the actuator shown in FIGS. 3 and 4B-4C. In particular, actuator 22′ is shown to have a distal portion 22a′, an intermediate portion 22b′ and a proximal portion 22c′ that extends to a proximal end opening 22f′. As shown, portion 22c′ is a frusto-conical portion that has an enlarged distal portion that tapers in the proximal direction. Distal portion 22a′ has a distal opening 22e′, such that a through passage extends between distal opening 22e′ and proximal end opening 22f′ for actuator 22′. By having frusto-conical section 22c′, membrane 20f′ of seal member 20′ may be more readily pierced or opened when seal member 20′ is biased or driven to shift axially in the distal direction by an impact force such as for example by the luer connector of a syringe or other similar connector coupling to the catheter hub, as per shown in FIG. 11C and described in the '104 US patent.

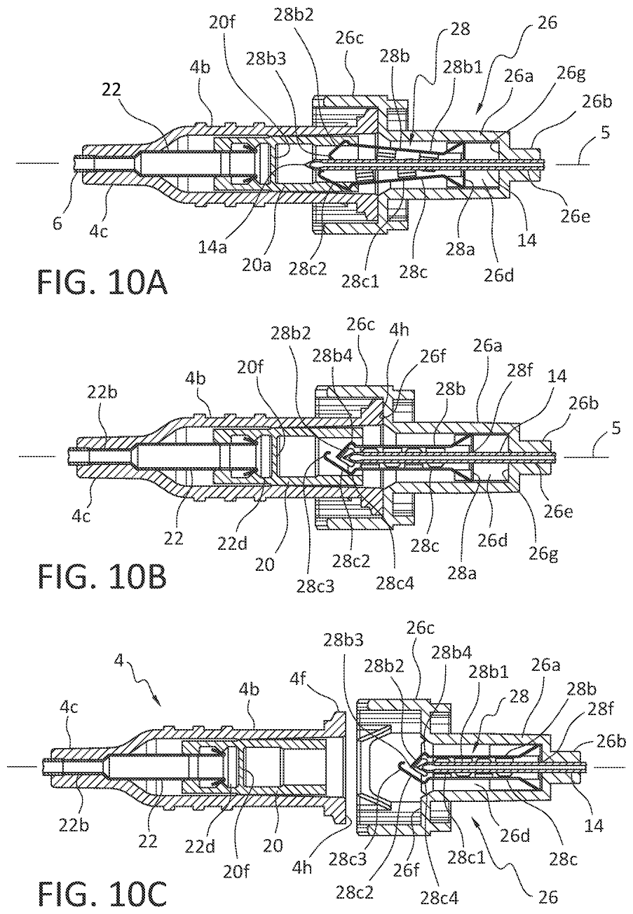

The safety catheter assembly includes a needle tip protector guard, or simply clip guard, that is fitted within the distal portion of the needle housing as shown in the ready to use, or simply ready position, in FIGS. 2 and 4B-4C. Clip guard 26 shown is similar to the clip protector guard described in the above-noted incorporated by reference '497 application. In particular, as shown in FIGS. 4B-4C and 10A-10C, the needle protector guard or clip guard 26 has a cylindrical center section 26a and a proximal section 26b that has a smaller diameter than cylindrical section 26a. At the distal end of center section 26a is a distal collar 26c that has a larger diameter than center section 26a. As best shown in FIGS. 10A-10C, an interior cavity 26d is defined within the wall of cylindrical center section 26a. An aperture 26e at proximal section 26d enables needle 14 to pass into and move relative to clip guard 26. Collar 26c is configured to have a dimension that allows it to be coupled to catheter hub 4 by way of the latter's flange 4f, as per shown FIGS. 10A-10B, which also show the proximal contact surface 4h of catheter 4 being in abutment contact with the back surface 26f of collar 26c of clip guard 26.

Mounted onto needle 14 is a needle tip protector or safety clip 28. As shown, needle tip protector or safety clip 28 has a proximal base 28a that has two elongated arms 28b and 28c connected thereto by bridging portions 28d and 28e, respectively. Bridging portions 28d and 28e each narrow similar to the top half of an hour glass, or Y-shaped, from the opposing side edges of base 28a to the respective arms 28b and 28c. Arms 28b and 28c are configured to extend substantially orthogonally from base 28a but at planes that are sandwiched by the imaginary planes of the opposing sides of base 28a when the arms are not being biased as per shown in their natural or unbiased position in FIGS. 10B-10C. The Y-shape configuration of bridging portions 28d and 28e provide more flexibility for arms 28b and 28c.

Arms 28b and 28c each have side ribs 28b1 and 28c1, respectively, that extend along the respective longitudinal sides of the arms. Ribs 28b1 and 28c1 are configured to be substantially orthogonal toward the longitudinal central axis of the safety clip 28. When in the position as shown in FIGS. 10B-10C, arms 28b and 28c together with their respective ribs 28b1 and 28c1 form a cage like enclosure. Barriers 28b2 and 28c2 are formed at the respective distal ends of arms 28b and 28c, with distal end barrier 28c2 overlapping distal end barrier 28b2 when the safety clip is in the closed or safe position as per shown in FIGS. 10B-10C. Although not shown, barrier 28c2 may have side ribs or partitions to protect the sharp distal tip 14a of needle 14 when needle 14 is withdrawn and enclosed or sheltered in safety clip 28. When thus sheltered, sharp distal tip 14a is encircled by the distal end barriers and the not shown side partitions. It should be appreciated that side partitions may be provided at barrier 28b2 instead of at barrier 28c2.

Barriers 28b2 and 28c2 have respective curved distal end or fingers 28b3 and 28c3. Rounded apexes, projections, protrusions or bosses 28b4 and 28c4 are formed at the respective junctions where arms 28b and 28c connect to their corresponding barriers 28b2 and 28c2. An opening 28f is formed at base 28a that has a diameter sized to enable a needle cannula such as needle 14 to pass through but prevents a needle feature that has a cross dimension different from the needle to pass as described in the incorporated by reference '497 application. The needle is adapted to pass between the one and other distal end barriers 28b2 and 28c2 when the barriers are positioned in the interior cavity of the catheter hub such that the barriers are biased in opposing directions away from the longitudinal axis 5 to effect a frictional engagement with the wall 20a at the proximal portion 20c of the seal member 20. Accordingly, clip guard 26 is fixedly held to the catheter hub 4 in the ready position as per shown in FIG. 10A. Safety clip 28 may be made from a metallic material such as stainless steel but with sufficient flexibility so that arms 28b and 28c may be biased scissor like from base 28a in a direction away the longitudinal axis 5, when the needle passes through and biases fingers 28b2 and 28c2 away from each other. Other embodiments of the clip guard may be gleaned form the '497 application.

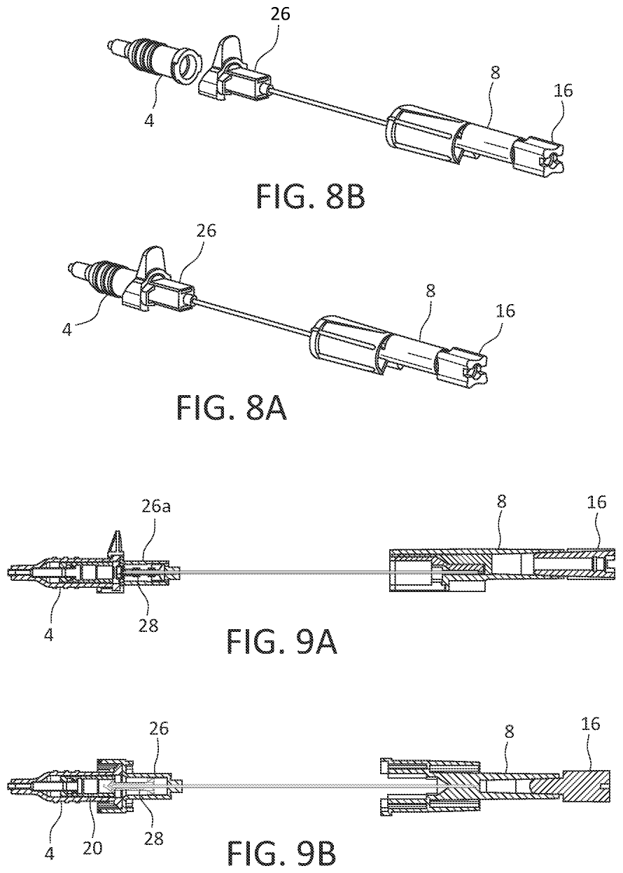

FIGS. 8A-8B and 9A-9B illustrate the safety catheter assembly in the safe position. More particularly, distal tip 14a of needle 14 is captured within safety clip 28 as per shown in FIGS. 10A-10C. In FIG. 10A, needle tip 14a is shown to bias barriers 28b2 and 28c2 as needle 14 is being withdrawn in the proximal direction. The needle feature, not shown, of needle 14 has not reached base 28a in FIG. 10A. Thus, base 28a of safety clip 28 is shown to be distally spaced from the back surface 26g of clip guard 26 in FIG. 10A. Since barriers 28b2 and 28c2 are being biased outwardly away from longitudinal axis 5 into wall 20a of seal member 20, safety clip 28 and clip guard 26 are fixedly held to catheter hub 4. In FIG. 10B, distal tip 14a of needle 14 has been moved to a position proximal of barriers 28b2 and 28c2 inside safety clip 28. Accordingly, arms 28b and 28c are no longer biased by needle 14 away from longitudinal axis 5. As a result, apexes 28b4 and 28c4 are released from wall 20a of seal member 20 such that safety clip 28 no longer is in frictional contact with proximal portion 20d of seal member 20. As needle 14 is further moved in the proximal direction and the needle feature comes into contact with base 28a, safety clip 28 is moved in the proximal direction so as to contact rear surface 26g of clip guard 26, such that clip guard 26 is moved proximally relative to catheter hub 4 and eventually separates from catheter hub 4, as per shown in FIG. 10C. FIGS. 8A and 8B correspond to FIGS. 10B and 10C in that the device is in the safe position as clip guard 26 is decoupled and separated, respectively, from catheter hub 4. FIGS. 9A and 9B are respective cross-sectional side view and top view of the safety catheter device in the safe position where safety clip 28 has moved into the cylindrical section 26a of clip guard 26.

With reference to FIGS. 11A-11C, the operation establishing a through fluid path from catheter 6 through catheter hub 4 and output from the proximal opening 24b of luer connector 24 is described. In brief, connector 24 has a central shaft 24a that has a bore 24a2 that connects proximal opening 24b to distal opening 24a1, which may also be considered as the distal end surface of shaft 24a. It should be noted that the actuator 22′ shown in FIG. 11A-11C is different from actuator 22 shown in FIGS. 10A-10C. In FIGS. 10A-10C, the actuator 22 has a flange or barb 22c at its proximal end positioned distal to the membrane of the seal member in the ready position. As described in the above-noted patent U.S. Pat. No. 8,652,104, when the seal member is impacted by a distal force, the membrane is biased against and pierced by the actuator. The membrane is held opened by the back side of the proximal barb when the impact force is removed. The actuator 22′ shown in FIGS. 11A-11C, on the other hand, has no flange or barb at its proximal end. Thus, when the impact force is removed from the seal member, due to the inherent resilient elasticity of the elastomeric seal member as well as the accordion shape of distal portion 20d′ of seal member 20′, seal member 20′ will return to its natural unbiased position such that membrane 20f′ reseals and is repositioned proximal to actuator 22′ as per shown in FIG. 11B. A circumferential lip 20h′ distal of membrane 20f′ acts as a seal for the proximal portion of actuator 22′ to prevent fluid from leaking in the distal direction from the distal portion 20d′ of seal member 20′.

Return to the operation whereby the fluid through passage is established between the catheter and the luer connector. As shown in FIG. 11c, connector 24 is moved in the distal direction as indicated by directional arrow 12, end surface 24a1 of shaft 24a thus comes into contact with proximal end 20g′ of seal member 20′ so that the seal member is moved in the distal direction to cause membrane 20f′ to bias against and be pierced by the proximal end 22f′ of actuator 22′. With the opening of membrane 20f′, a through fluid path is established as per shown in FIG. 11C. Detailed discussion of the interaction between the seal member and the luer connector is given in the above-noted '104 US patent.

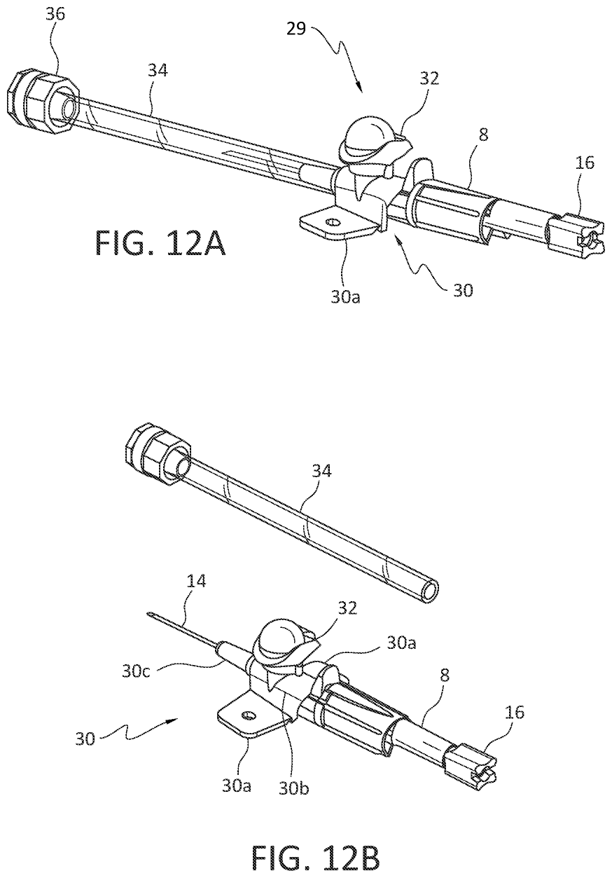

FIG. 12A shows a safety catheter assembly 29 that has a catheter hub 30 having an upright sideport 32 integrally extending from catheter hub 30. Needle 14 is covered by a sheath 34 that has a luer lock cap 36. Safety catheter assembly 29 has the same needle housing 8 and flash plug 16 as in the embodiments discussed above and thus have the same labeled components. Catheter hub 30 is shown to have a pair of wings 30a extending from its base. FIG. 12B shows sheath 34 removed from needle 14 and distal portion 30c of catheter hub 30.

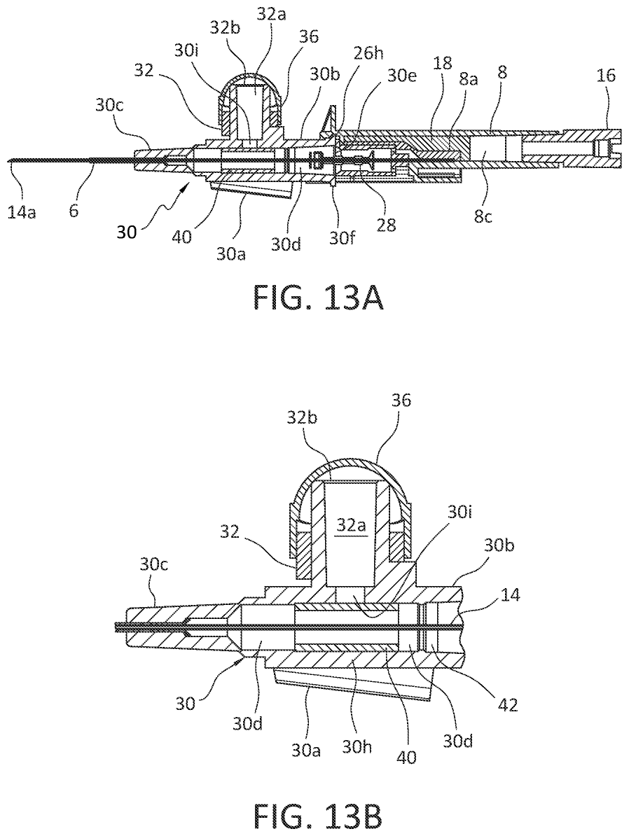

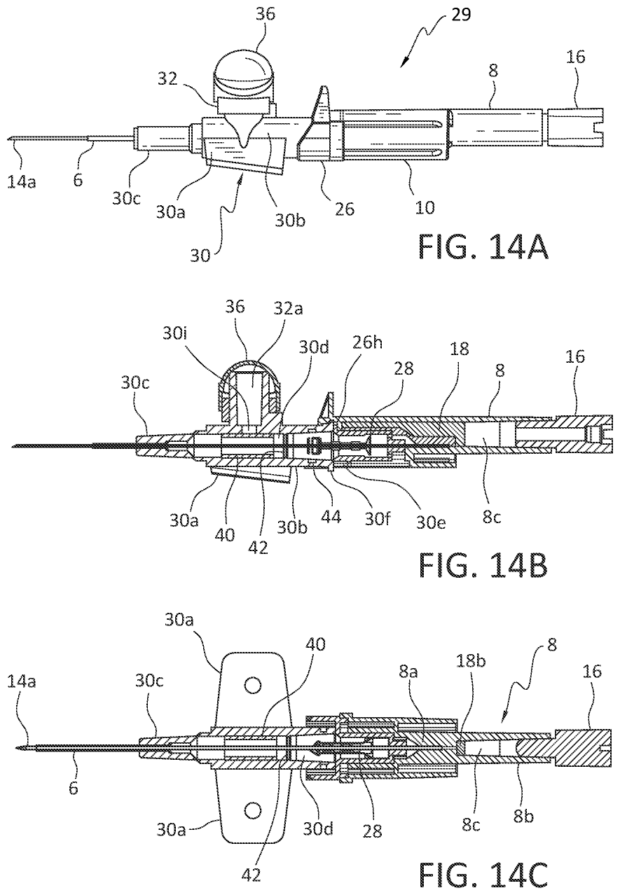

As shown in FIGS. 13A-13B and 14A-14C, the proximal end 30e of catheter hub 30 is coupled to clip guard 26. For the embodiment shown, clip guard 26 may have an internal notch 26h dimensioned to accept flange 30f of catheter hub 30 in a snap fit configuration. It should further be appreciated that a locking feature such as for example an internal rib, protuberance or groove 44 may be provided in the inner surface of the proximal portion of catheter hub 30 to frictionally contact or coact in an interference fit with the apexes or bumps of the barriers of the arms of the safety clip when the arms of the safety clip are biased away from each other or from the longitudinal axis of the needle assembly by the needle passing therethrough, as discussed above. Thus, for the embodiment shown in FIGS. 13A-13B, even though there is no seal member in the catheter hub in the manner of the embodiments described in FIGS. 2-10, the clip guard nonetheless may be fixedly held to the catheter hub so long as the arms of the safety clip are being biased by the needle.

Be it a snap fit or an interference fit, as with the earlier embodiment described in FIGS. 4A-4C, a blood diverter 18 is provided in needle housing 8 that includes flashback chamber 8c. Needle housing 8 and blood diverter 18 therein are the same as those disclosed in the FIGS. 4A-4C embodiment. For the FIGS. 13A-13B and 14A-14C embodiment where the catheter hub 30 has a sideport 32, the bore 32a of sideport 32 is separated from the interior cavity 30d of catheter hub 30 by an elastomeric cylindrical seal member 40 located in the interior cavity 30d to cover bore 32a.

With reference to the enlarged cross-sectional view of FIG. 13B, catheter hub 30 includes a main portion 30b and a distal portion 30c. Catheter hub 30 has a body defined by a wall 30h that forms an interior or internal cavity 30d. Sideport 32 integrally extends outwardly from the main body portion 30b. Sideport 32 has a passage or bore 32a that extends into interior cavity 30d via an opening 30i at wall 30e to establish a fluid communication path between the internal cavity of the catheter hub 30 and sideport 32. A cylindrical seal member 40 which may be made from a flexible resealable elastomeric material such as silicone or polyisoprene is fitted in the portion of interior cavity 30d to block opening 30i. As is conventionally known, with cap 18 removed, the open end 32c of sideport 32 may be connected to a fluid store such as a syringe or a fluid injection pump, possibly by means of a tubing, so that fluid under pressure may be injected into sideport 32. Seal member 40 is responsive to a given pressure such that when the fluid injected into sideport 32 exceeds the given pressure, seal member 40 opens to establish a through fluid path between the fluid store and internal cavity 30d of catheter hub 30, and from there through catheter 6 to the vein or artery of the patient.

For the illustrations in FIGS. 13A-13B and 14A-14C, reference number 42 may represent an internal groove in the catheter hub that a septum may be fitted to prevent fluid in interior cavity 30d of catheter hub 30 from escaping in the proximal direction. Such scenario may happen when needle 14 has been removed from the catheter hub with the open proximal end of catheter hub 30 not connected to a fluid store device so that proximal end 4e is opened and fluid is injected under pressure from sideport 32 to open seal member 40 such that the fluid flows into the interior cavity 30d.

Sideport 32 integrally extends as an upright from catheter hub 30. Sideport 32 is shown being covered by a snap on cap 36 in FIGS. 14A-14B. Same as with the earlier embodiment, flash plug 16 is attached to the open proximal end 8b of needle housing 8 to enable the clinician to monitor blood at the flashback chamber 8c of needle housing 8 to determine whether the needle, and the catheter that overlies the needle, are correctly placed into the vasculature of the patient as conventionally known. One continuous flange/rib or multiple protuberances, ribs or flanges 30f integrally extend transversely from the proximal end 30e.

As shown in FIG. 14B-14C, the sharp distal tip 14a of needle 14 extends beyond the distal end of catheter 6. The proximal or non-patient end 14b of needle 14 is fixedly attached to a needle hub 6c of needle housing 6. As discussed above, the needle shaft defines a lumen and may have a side orifice (not shown) proximate to its distal tip as is conventionally known to enable fluid to flow into the lumen and egress at the orifice into a space defined between the outer wall of the needle and the inner wall of the catheter, and the fluid may flow into the flashback chamber in needle housing 8 as is also conventionally known.

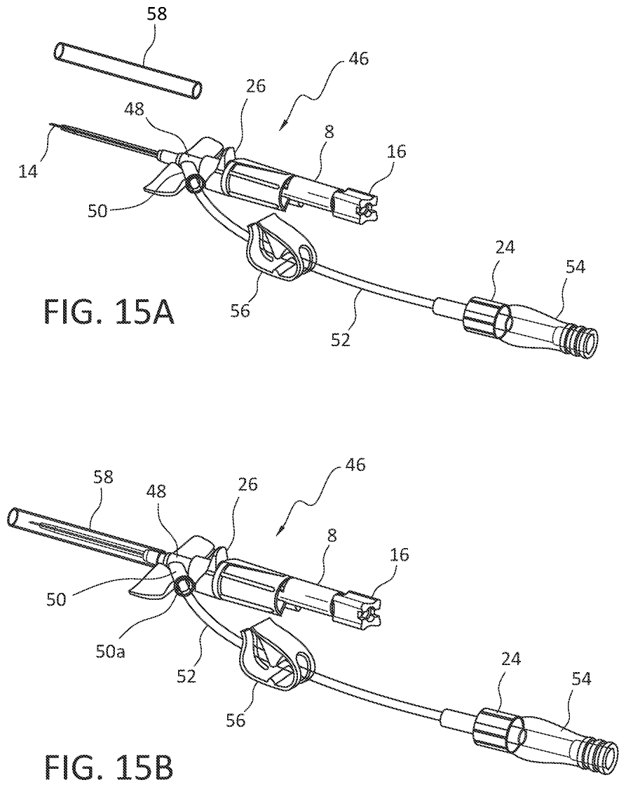

FIGS. 15A-15B are illustrations of a closed system catheter (CSC) needle assembly 46 where its catheter hub 48 includes a sideport 50. The needle housing 8, the flash plug 16 and the clip guard 26 for the CSC needle assembly are the same as described in the earlier embodiments. As shown, a tubing 52 is connected to the open end 50a of sideport 50 at one end and a luer connector 24 at its other end. As is conventionally known, connector 24 may be connected to a fluid store including for example a syringe, pump, injector or the like. A clip 56 slidable along tubing 52 is used to occlude the tubing to shut off the flow of fluid along tubing 52 when needed. Needle 14 is covered by a sheath 58 before use. As shown in FIGS. 15A-15B, the CSC needle assembly is in the ready position.

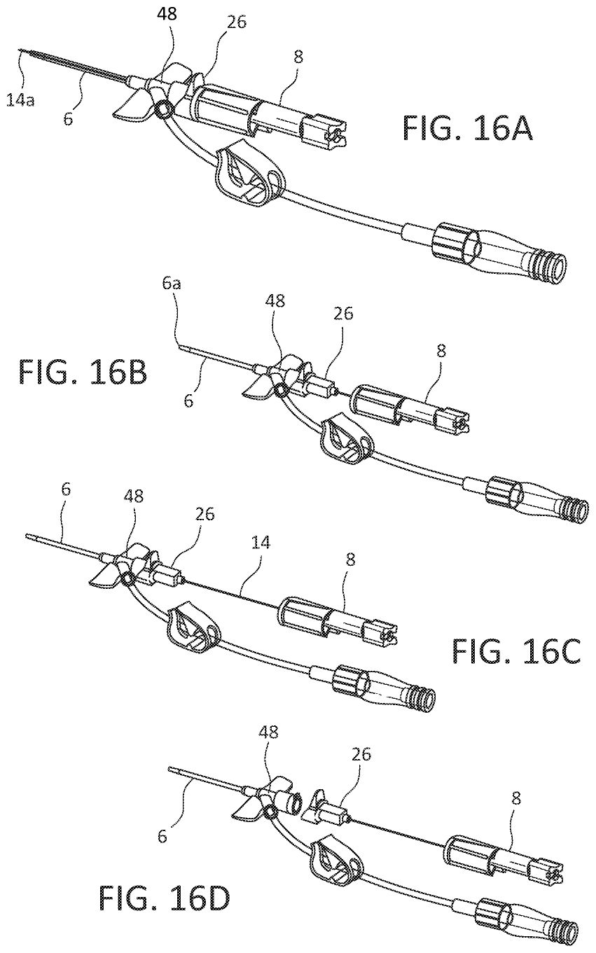

The various operational states that the CSC needle assembly 46 may be in are shown in FIGS. 16A-16D. FIG. 16A shows the CSC needle assembly in the ready to use state where the clip guard 26 and catheter hub 48 are coupled together, with clip guard 26 housed in the distal portion of needle housing 8. FIG. 16b shows an advancing state where the sharp distal tip 14a of needle 14 and the distal end of catheter 6 are placed into the vasculature of the patient, and that the clinician is advancing the distal end of the needle further into the vein or artery of the patient while needle housing 8 is held. FIG. 16C shows that catheter 6 has been advanced into the vein or artery of the patient, and needle 14 is being withdrawn from catheter hub 48 such that the sharp distal tip 14a is moved into the safe position inside safety clip 28 as described above. FIG. 16D shows the separation of clip guard 26 from catheter hub 48 such that the needle assembly is separated from the CSC catheter hub assembly.

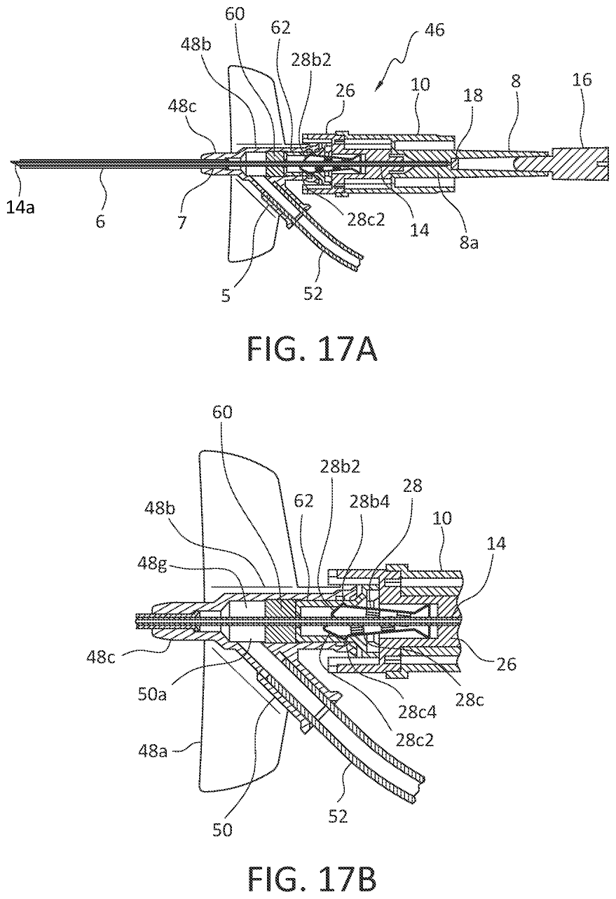

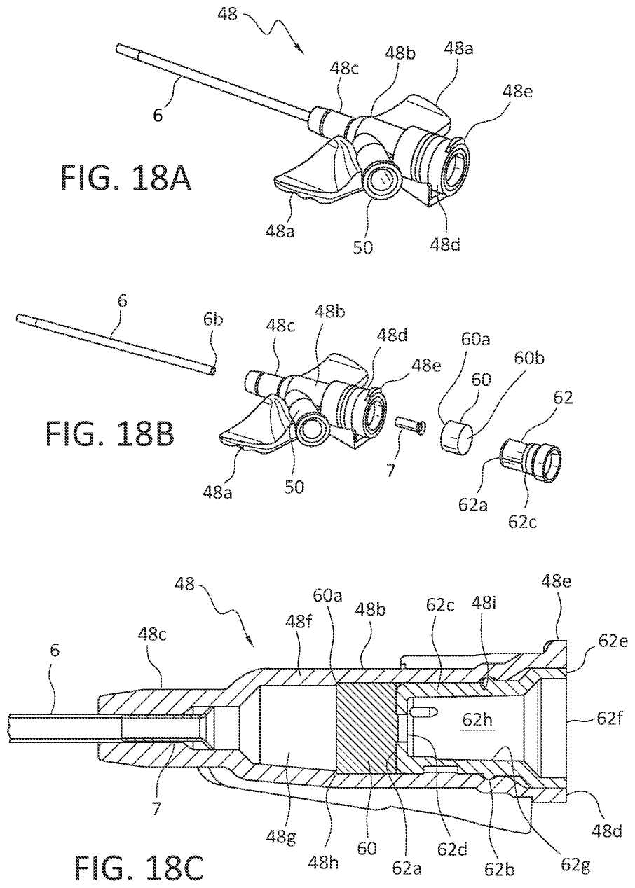

FIGS. 17A-17B and 18A-18C show an exemplar embodiment of the CSC needle assembly in which a septum retainer is added to the catheter hub as described in U.S. Pat. No. 10,675,440, assigned to the same assignee as the instant application. The disclosure of the '440 patent is incorporated by reference in its entirety to the disclosure of the instant application.

With reference to FIGS. 17A-17B and 18A-18C. the CSC catheter hub 48 includes a main body portion 48b having a pair of wings 48a extending therefrom, a distal portion 48c and a proximal portion 48d. A protuberance 48e at proximal portion 48d allows the catheter hub 48 to be coupled to clip guard 26. An interior cavity 48g is defined by the wall 48f of catheter hub 48. As discussed above, proximal end 6b of catheter 6 is connected to an aperture in distal portion 48c and is held thereat by an eyelet 7 press fitted into the aperture, as shown in FIG. 18C. The different components of catheter hub assembly 48 are separately shown in FIG. 18B. An internal circumferential step 48h at the inner surface of wall 48f in contact with the distal edge 60a of septum 60 prevents distal movement of septum 60 relative to catheter hub 48.

A septum retainer 62 is inserted into the proximal portion of catheter hub 48 via open proximal end 48d of catheter hub 48 such that the distal end surface 62a of septum retainer 62 abuts the proximal end surface 60b of septum 60, so that septum 60 is held in place inside catheter hub 48. A circumferential groove 48i at the inner surface of wall 48f accepts an external circumferential protuberance 62b at the outer surface of wall 62c of septum retainer 62 to fixedly hold septum retainer 62 to the proximal portion of catheter hub 48. An aperture 62d is provided at distal end 62a of septum retainer 62 to enable needle 14 to pass through to pierce septum 60 and slidably extends along catheter 6, as per shown in FIG. 17A. A proximal opening 62f is provided at proximal end 62e, which may be flush with proximal end 48d of catheter hub 48. Proximal opening 62f may also be distal of proximal end 48d. An internal or interior cavity 62h is defined by the inner surface 62g of wall 62c of septum retainer 62. As best shown in FIG. 17B, sideport 50 is fluidly communicative with interior cavity 48g of catheter hub 48 through an opening 50a distal from septum 60.

The needle assembly used with CSC catheter assembly shown in FIGS. 18A-18C is the same as the needle assembly discussed in the earlier embodiments. For the embodiment shown in FIGS. 17A-17B, at least the distal portion of safety clip 28 is inside the internal cavity 62h of septum retainer 62 in the ready position. That is, the distal portions of arms 28b and 28c, the barriers 28b2 and 28c2 and the protuberances or apexes 28b4 and 28c4 of the arms of safety clip 28 are located in cavity 62h of septum retainer 62 in the ready position. As discussed above, needle 14, in the ready position, biases the arms 28b and 28c by means of the needle's contact with barriers 28b2 and 28c2. As a result, the respective apexes 28b4 and 28c4 are biased into frictional contact with the inner surface 62g of wall 62c of septum retainer 62. As a result, clip guard 26 is fixedly held to catheter hub 48 since catheter hub 48 and septum retainer 62 are fixedly held to each other. As per discussion above, when the distal tip 14a of needle 14 is withdrawn in the proximal direction relative to catheter hub 48 such that barriers 28b2 and 28c2 are no longer biased by needle 14 away from the longitudinal axis of the needle assembly, the barriers would return to their natural unbiased position to block distal tip 14a of needle 14 from further distal movement as well as to release the apexes 28b4 and 28c4 from the inner surface 62g of septum retainer 62. Clip guard 26 may then be separated from catheter hub 48, as per shown in FIG. 16D.

It is the intension of the inventors that all matter described throughout this specification and shown in the accompanying drawings be interpreted as illustrative only and not in a limiting sense. Accordingly, it is intended that the invention be limited only by the spirit and scope of the hereto appended claims.

1. Apparatus comprising: a needle housing having a needle hub with a passageway, a needle having a sharp distal end and a proximal end connected to the needle hub, the needle having a lumen extending from the sharp distal end to the proximal end in alignment with the passageway of the needle hub, a rear chamber in the needle housing proximal the needle hub adapted to receive fluid that enters the distal end of the needle, and a fluid diverter in fluid communication with the needle hub for receiving the fluid incoming from the lumen and redirecting the fluid to flow to a distal section of the fluid diverter viewable through a distal portion of the housing.

2. The apparatus of claim 1, wherein the fluid diverter is further configured to guide the fluid from the distal section to the rear chamber, the rear chamber being a flashback chamber that enables a user to see the fluid.

3. The apparatus of claim 1, wherein the needle housing is an elongate tubular member having an open proximal end, the needle hub integrally formed at the distal portion inside the elongate tubular member, the open proximal end fitted with a flash plug that allows air to pass but prevents the fluid from passing.

4. The apparatus of claim 3, wherein a gripper member adapted to be gripped by a user integrally and semi-circumferentially extends from the distal portion of the needle housing.

5. The apparatus of claim 1, wherein the needle housing is transparent or translucent such that the presence of fluid can been observed at the distal portion and the rear chamber of the needle housing.

6. The apparatus of claim 1, wherein the fluid diverter is configured to reroute the fluid in the distal section to the rear chamber of the needle housing.

7. The apparatus of claim 1, wherein the fluid diverter comprises an enclosure having a bore in fluid communication with the needle hub such that the incoming fluid is directed to the bore, the bore leads to a distal direction fluid path that redirects the incoming fluid to flow in a distal direction to the distal section of the enclosure, the fluid diverter further having at least one proximal direction fluid path in fluid communication with the distal section such that the fluid at the distal section is rerouted in a proximal direction to the rear chamber, the rear chamber being a flashback chamber.

8. The apparatus of claim 7, wherein the distal direction fluid path is separated into two proximal direction fluid paths at the distal section for rerouting the fluid to the flashback chamber.

9. The apparatus of claim 7, wherein the fluid diverter has a distal surface in abutment with a proximal surface of the needle hub such that the passageway of the needle hub is in alignment with the bore of the fluid diverter.

10. The apparatus of claim 4, wherein the gripper member comprises two semi-circular wings sandwiching at least the distal portion of the needle housing that encloses the distal section of the fluid diverter, the needle housing being transparent or translucent so that the user may, based on observation of fluid flowing into the distal section of the fluid diverter, determine that the distal sharp tip of the needle is correctly placed into the vein or artery of a patient.

11. The apparatus of claim 1, further comprising:
a catheter hub having a distal end and an open proximal end, an internal cavity defined between the distal end and the open proximal end of the catheter hub;
wherein the distal portion of the needle housing includes a coupler for connecting to the open proximal end of the catheter hub such that the needle extends through the catheter hub and a catheter extending from the distal end of the catheter hub with the distal end of the needle extending beyond a distal end of the catheter.

12. A method of establishing blood flow in a needle housing, comprising the steps of:
providing a needle housing including a distal portion having a transparent or translucent viewing part;
attaching a proximal end of a needle having a lumen and a sharp distal end to a needle hub integrally formed inside a distal portion of the needle housing such that the lumen of the needle is in alignment with a passageway of the needle hub to establish a fluid passage for receiving blood that enters the distal end of the needle when the needle is placed into a vein or artery of a patient;
providing a flashback chamber in the needle housing proximal the needle hub; and
positioning a fluid diverter adjacent the needle hub for receiving the blood output from the passageway, the fluid diverter redirecting the blood to flow in a distal direction to the transparent or translucent viewing part of the distal portion of the housing where a user can observe the blood flow.

13. The method of claim 12, further comprising the step of:
configuring the fluid diverter to guide the blood from the distal portion of the needle housing to the flashback chamber where the user can see the blood.

14. The method of claim 12, wherein the needle housing is an elongate tubular member having an open proximal end, further comprising the step of:
fitting a flash plug that allows air to pass but prevents the fluid from passing to the proximal open end of the elongate tubular member.

15. The method of claim 12, further comprising the steps of:
configuring the fluid diverter to have an enclosure having a bore in fluid communication with the passageway of the needle hub such that blood from the needle flows into the bore; and
configuring the bore to have a distal direction fluid path that redirects the blood from the passageway to flow in a distal direction to a distal section of the fluid diverter, the distal section in fluid communication with at least one proximal direction fluid path such that the blood at the distal section of the fluid diverter is rerouted in a proximal direction to the flashback chamber.

16. The method of claim 16, further comprising the step of:
separating the distal direction fluid path into two proximal direction fluid paths at the distal section for rerouting the fluid to the flashback chamber.

17. Apparatus, comprising:
a needle housing including a proximal portion, a distal portion having a transparent or translucent viewing part and a flashback chamber at the proximal portion;
a needle having a lumen, a sharp distal end and a proximal end attached to a needle hub integrally formed inside a distal portion of the needle housing such that the lumen of the needle is in alignment with a passageway of the needle hub to establish a fluid passage for receiving blood that enters the distal end of the needle when the needle is placed into a vein or artery of a patient; and
a fluid diverter positioned adjacent the needle hub for receiving the blood output from the fluid passage, the fluid diverter redirecting the blood to flow in a distal direction to a distal section of the fluid diverter located in the transparent or translucent viewing part of the distal portion of the housing where a user can observe the blood flow.

18. The apparatus of claim 17, wherein the fluid diverter is configured to reroute the fluid in the distal section to the flashback chamber.

19. The apparatus of claim 17, wherein the fluid diverter comprises an enclosure having a bore in fluid communication with the needle hub such that the incoming fluid is directed to the bore, the bore leads to a distal direction fluid path that redirects the incoming fluid to flow in a distal direction to the distal section of the enclosure, the fluid diverter further having at least one proximal direction fluid path in fluid communication with the distal section such that the fluid at the distal section is rerouted in a proximal direction to the flashback chamber.

20. The apparatus of claim 19, wherein the distal direction fluid path is separated into two proximal direction fluid paths at the distal section for rerouting the fluid to the flashback chamber.
